(19) United States
(12) Patent Application Publication
(10) Pub. No.: US 2022/0049287 A1
(43) Pub. Date: Feb. 17, 2022

(54) Colorimetric sensing polymerase chain reaction (PCR)

(71) Applicant: GM Biosciences, Inc, Frederick, MD (US)

(72) Inventors: Guozhi Zhu, Frederick, MD (US); Miao Qiao, Frederick, MD (US)

(73) Assignee: GM Biosciences, Inc, Frederick, MD (US)

(21) Appl. No.: 17/275,317

(22) PCT Filed: Sep. 13, 2019
(86) PCT No.: PCT/US19/50982

§ 371 Date: Mar. 11, 2021

Related U.S. Application Data
(60) Provisional application No. 62/733,853, filed on Sep. 20, 2018.

(51) Int. Cl. C12Q 1/686

(52) U.S. Cl. CPC C12Q 1/686

(57) ABSTRACT

Methods are provided here for an easy and reliable non-fluorescent approach to detecting and monitoring PCR amplification. This includes monitoring PCR amplification by using magnesium-sensitive colorimetric dye. The dye is visually detectable color dye. Compositions are described here that comprise a magnesium-sensitive colorimetric dye, a buffer, dNTPs, magnesium ion and DNA polymerase.

### BACKGROUND OF INVENTION

According to the ways of manipulating temperature, nucleic acid amplification methods fall into two categories: polymerase chain reaction (PCR) which relies on thermal cycling (repeated heating and cooling), and isothermal amplification which is carried out at a constant temperature.

The most popular ways to analyze PCR product are gel electrophoresis and real time fluorescent analysis, which needs labor intensive manual process or expensive instrumentation. In this respect, colorimetric method is very attractive in which PCR product can be detected by the unaided eyes without laborious process and expensive instrumentation. For examples, Fe²⁺/Fe²⁺ indicator (Lee, et al., Biotechnol Lett., 20:1739-42 (2003)), G-quadruplex/hemic complex (Bhadra, et al., Anal Biochem., 445: 38-40 (2014)), gold nanoparticles (Valentini, et al., Angew Chem Int Ed Engl., 55(6): 2157-60 (2016)) were added into PCR products right after PCR completion, and then colors were developed and detected by naked eyes. Most of such agents, such as heavy metal ion (such as Fe²⁺) and hemin, have to be added into post-amplification PCR product because they are widely known to inhibit PCR reaction (Schrader, et al., J Appl Microbiol., 113(5):1014-26 (2012)). However, when opening a post-PCR tube to add such agents into it, tiny droplets of aerosolized PCR products easily spread all over and contaminate the working area, which will give rises to false positives for future testing. Ideally, the post-PCR tube keeps closed and the colorimetric agent is added in advance before initiation of PCR reaction. Recently, New England BioLabs (NEB) presents such a method, in which a pH-sensitive dye is added into a weak-buffered PCR solution prior-to-reaction (Tanner, et al., Biotechniques, 58(2): 59-68 (2015)). PCR produces hydrogen ion to lower the pH which is then monitored by a pH-sensitive dye. The pH-sensitive dye changes its color in response to pH change, which is then visually detected by naked eye. Laboratory of Cai reported another prior-to-reaction colorimetric method in which primers were conjugated on Gold nanoparticle and PCR amplification resulted in aggregation of nanoparticle (Cai, et al., Nano Res., 3:557-63 (2010)). Aggregation of gold nanoparticle gave rise to color change from red to pink/purple.

LAMP (loop mediated isothermal amplification), an isothermal amplification technique, can be visually detected by prior-to-reaction means, such as pH-sensitive dye (Tanner, et al., Biotechniques, 58(2): 59-68 (2015)), and magnesium-sensitive dye (e.g. Eriochrome Black T, EBT; Hydroxynaphthol Blue, HNB) (Wang, et al. Applied Mechanics and Materials, 618: 264-7 (2014); Rodriguesz-Manzano, et al., ACS Nano., 10(3): 3102-13 (2016); Goto, et al., Biotechniques, 46(3): 167-72 (2009)). DNA amplification in LAMP produces tremendous pyrophosphate, which forms magnesium pyrophosphate (MgPpi) precipitates. The formation of MgPpi reduces magnesium ion in solution (Wang, et al. Applied Mechanics and Materials, 618: 264-7 (2014)). The concentration of magnesium is monitored by magnesium-sensitive dye EBT and HNB. The reduction of magnesium concentration induces color change of HNB and EBT (Rodriguesz-Manzano, et al., ACS Nano., 10(3): 3102-13 (2016)).

Although magnesium-sensitive dyes are successfully employed in LAMP, but there is no evidence showing that they have been used in PCR. The current invention presents ways of using magnesium-sensitive dyes in PCR.

### SUMMARY

In an embodiment of the invention, a preparation of PCR solution is provided that comprises magnesium-sensitive colorimetric dye, buffer, magnesium divalent ion (Mg²⁺), primers, DNA polymerase and dNTPs. In one aspect, the preparation includes template DNA. In another aspect, the magnesium-sensitive colorimetric dye is a visually detectable dye. In another aspect, the magnesium-sensitive colorimetric dye is EBT. In another aspect, the magnesium-sensitive colorimetric dye is HNB.

In some embodiments of the invention, a post-PCR reaction comprising a magnesium-sensitive colorimetric dye, wherein the dye is added before initiation of PCR or after PCR completion. In another aspect, the magnesium-sensitive colorimetric dye is a visually detectable dye. In another aspect, the magnesium-sensitive colorimetric dye is EBT. In another aspect, the magnesium-sensitive colorimetric dye is HNB.

In one embodiment of the invention, a method for detecting PCR amplification comprising: providing a PCR amplification reaction mixture comprising a magnesium-sensitive colorimetric dye, a buffer, magnesium ion, a DNA polymerase, dNTPs, primers and template DNA; and detecting a color change of the dye resulting from amplification of the target DNA. In another aspect, the magnesium-sensitive colorimetric dye is a visually detectable dye. In another aspect, the magnesium-sensitive colorimetric dye is EBT. In another aspect, the magnesium-sensitive colorimetric dye is HNB. In another aspect, the dye is added before initiation of PCR. In another aspect, the color of the dye is monitored during PCR reaction. In one aspect, the method further comprises quantification of template DNA by threshold number of cycles. In another aspect, the method further comprises comparing a color change of the magnesium-sensitive dye from before to after the PCR reaction. In another aspect, the method further comprising comparing the colors of PCR reactions with template to that of PCR reactions without template. In another aspect, the dye is added after PCR completion. In another aspect, the method further comprising comparing the colors of PCR reactions with template to that of PCR reactions without template.

### BRIEF DESCRIPTION OF FIGURES

The patent or application file contains at least one figure executed in color. Copies of this patent or patent application publication with color figures will be provided by the Office upon request and payment of the necessary fee.

FIG. 1 shows the visible colors of magnesium-sensitive dye EBT in PCR preparation solution with various amount of Mg²⁺. PCR preparation solutions were prepared, which included buffer, DNA polymerase, dNTPs, primers, EBT and various amount of Mg²⁺ (from left to right: 0.5 mM, 1 mM, 1.5 mM, 2 mM, 2.5 mM, 3 mM). Without performing PCR reaction, photo was directly taken under visible light. With the increase of Mg²⁺, the colors of samples turned from blue to purple.

FIG. 2 shows PCR amplification detected by magnesium-sensitive dye EBT. EBT was added into reactions after PCR completion. The color change was specific to amplification of target DNA.

FIG. 2A. Two PCR reactions with (left) or without (right) template DNA were performed. After PCR completion, magnesium-sensitive dye EBT was added into each tube respectively. The color difference between two samples indicates that the reaction with template DNA has lower Mg²⁺ than the one without template. The Mg²⁺ reduction is detectable by magnesium-sensitive dye EBT.

FIG. 2B shows the gel electrophoresis of the same PCR products from FIG. 2A. The blue color in FIG. 2A was specific to amplification of target DNA.

FIG. 3 shows the colors of PCR reactions before PCR initiation and after PCR completion. EBT was added into PCR preparation solution in advance before initiation of PCR reaction.

FIG. 3A shows the color of PCR preparation solution before initiation of PCR reaction. EBT was added prior-to-reaction. PCR preparation solution contained buffer, KOD DNA polymerase, Mg²⁺, primers, dNTPs, magnesium-sensitive dye EBT, and with (left) or without (right) template. Both solutions exhibited purple color.

FIG. 3B shows the color of PCR product after PCR completion. The color of positive reaction (left) changed its original purple color to blue. The color of non-template reaction (right) kept its original purple color.

FIG. 3C shows the gel electrophoresis of the same PCR products from FIG. 3B.

FIG. 4A-4B shows the color change in PCR reactions with various amount of template DNA. EBT was added into PCR preparation solution in advance before initiation of PCR reaction. FIG. 4A shows the color of PCR reactions with various amount of template DNA after PCR completion. PCR preparation solutions containing EBT and various amount of template DNA were subjected to PCR reaction. After PCR completion, photo was taken. Tubes 1-4 (from left to right) represent 10⁸ copies, 10⁶ copies, 10⁴ copies and zero copy of template DNA. As shown, all reactions containing template DNA showed blue color, whereas the reaction with zero template showed purple color.

FIG. 4B shows the gel electrophoresis of the same PCR products from FIG. 4A. The reactions containing template DNA had robust amplifications, which were consistent to their color change shown in FIG. 4A. The color change thus clearly indicates the amplification of template DNA.

FIG. 5 Employment of magnesium-sensitive dye HNB in PCR. HNB was added before initiation of PCR.

FIG. 5A shows the color of PCR preparation solution before initiation of PCR reaction. PCR preparation solution contained magnesium-sensitive dye HNB, buffer, KOD DNA polymerase, Mg²⁺, primers, dNTPs, and with template DNA (left) or no-template control (right). HNB exhibited violet color before initiation of PCR reaction.

FIG. 5B shows the color of reactions after PCR completion. The color of positive reaction (left) changed its original violet to sky-blue color.

FIG. 5C shows the gel electrophoresis of the same PCR products from FIG. 5B.

FIG. 6 shows color of PCR reactions using Taq DNA polymerase. PCR preparation solution contained EBT, buffer, 3.5 mM Mg²⁺, primers, dNTPs, and with template DNA (left) or no-template control (right). Taq DNA polymerase was added to each tube during the initial denature step of PCR (95° C. 2 min).

FIG. 6A shows the color of reactions after PCR completion. After PCR completion, the photo was taken. As shown, positive reaction exhibited violet color whereas the negative reaction exhibited purple color.

FIG. 6B shows the gel electrophoresis of the same PCR products from FIG. 6A.

### DETAIL DESCRIPTION OF EMBODIMENTS

Mg²⁺ indicators such as HNB and EBT are ionochromic dyes that undergo a definite color change in presence of Mg²⁺ metal ions. They form a complex with the Mg²⁺ ions in the solution, which results in a color change. For example, the color of EBT alone is blue, and it turns red when it forms a complex with Mg²⁺. Both HNB and EBT are colorimetric indicator of Mg²⁺ ions. The term “Mg²⁺ indicator” may be used interchangeably with the term “magnesium indicator”, “magnesium-sensitive dye”, “Mg²⁺ sensitive dye”.

Mg²⁺ ion acts as a cofactor for DNA polymerase. Without it, DNA polymerase is inactive and PCR reaction cannot proceed. In LAMP system, 8 mM Mg²⁺ are provided to drive the reaction and the level of Mg²⁺ is reduced resulting from MgPpi precipitates. The reduction is detected by magnesium-sensitive dye such as HNB or EBT (Wang, et al. Applied Mechanics and Materials, 618: 264-7 (2014); Rodriguesz-Manzano, et al., ACS Nano., 10(3): 3102-13 (2016); Goto, et al., Biotechniques, 46(3): 167-72 (2009)). The same mechanism may also be applicable to PCR amplification. However, a typical PCR reaction needs an optimal 1.5 mM-2 mM Mg²⁺, which is much lower than 8 mM Mg²⁺ typically used in LAMP. It is unknown that whether PCR amplification results in reduction of Mg²⁺, and if it does, whether the reduction is sufficient to be detectable by EBT or HNB.

Embodiments of the invention provide compositions and methods for detection of PCR amplification using a Mg²⁺ indicator. Magnesium-sensitive dye senses various Mg²⁺ concentrations in PCR preparation solution containing 1× buffer, Mg²⁺, dNTPs, primers, DNA polymerase. In one exemplary embodiment, robust color change of EBT was observed, from sky-blue to blue, further to violet, and to purple. 0.5 mM Mg²⁺ difference in the range of 0.5 mM-2 mM was successfully detected by EBT (Example 1). In some exemplary embodiments, the reduction of Mg²⁺ concentration in PCR reaction was sufficient to change the color of magnesium indicator (Example 2, 3, 4, 5, 6, 7). In some exemplary embodiments, the reduction of Mg²⁺ was successfully detected by EBT with color change from original purple to blue or bluish (Example 2, 3, 4, 5, 7). In one exemplary embodiment, the reduction of Mg²⁺ was successfully detected by HNB with color change from violet to blue (Example 6). In some exemplary embodiments, PCR products were further analyzed by agarose gel electrophoresis (Example 2, 3, 4, 7), which showed that color change of EBT correlates to PCR amplification. In one exemplary embodiment, PCR products were analyzed by agarose gel electrophoresis (Example 6), which showed that color change of HNB correlates to PCR amplification. Therefore, PCR amplification indeed results in reduction of Mg²⁺, which is enough to become detectable by Magnesium-sensitive dye.

The employment of two magnesium-sensitive dyes, EBT and HNB, is not intended to be limiting. Other magnesium-sensitive dyes are also suitable for the present invention. The principle of the invention applies to other magnesium-sensitive dyes as well. The magnesium-sensitive dyes mentioned above can be chemically modified to have altered colorimetric properties in response to Mg²⁺ concentration change. These modifications can create dyes that are either brighter or change color responsive to a narrower Mg²⁺ change and thus allow a more sensitive detection.

The magnesium-sensitive dye is added either before the start of PCR reaction, during PCR reaction, or after the completion of PCR reaction. In one exemplary embodiment, EBT was added into post-PCR product (Example 2). In other exemplary embodiments, magnesium-sensitive dye was added before the initiation of PCR reaction (Example 3, 4, 5, 6, 7).

The color of dye is detected and monitored by many means. Examples include, but are not limited to, the eyes of the operator, a colorimeter, or a spectrophotometer. In some exemplary embodiments, the color of dye was detected by naked eyes. Instruments can detect the color change with much higher sensitivity.

The color of dye is detected in real-time manner during the amplification process, or at the endpoint of amplification. In one exemplary embodiment, the color of dye was detected in a real-time manner (Example 5). In other exemplary embodiments, the color of dye was detected at the endpoint of amplification (Example 2, 3, 4, 6, 7). Real time detection of color change, especially by instruments, can quantify the amount of template DNA, and thus allows quantitative detection of DNA.

Embodiments of the invention provide methods and compositions that detect PCR amplification by using magnesium-sensitive dyes under a variety of conditions. Detecting PCR amplifications by magnesium-sensitive dye were successfully performed in a series of exemplary embodiments with different set of primers, different magnesium-sensitive dyes, different amount of template DNA, different DNA polymerases, different concentration of Mg²⁺. The series of exemplary conditions mentioned above are not intended to be limiting.

In certain embodiment, the primer is completely reverse-complementary to template. In other certain embodiment, the primer is only partially reverse-complementary to template. For example, without any limitation, the primer may only have its 3′ partial region reverse-complementary to template. The 5′ region of primer may or may not be reverse-complementary to template. The 5′ region of primer may comprise restriction enzyme cutting site, nicking enzyme cutting site, enzyme binding site, or modified nucleic acids.

In certain embodiments, the template nucleic acid is double stranded or single stranded. The template nucleic acid may be, for example, selected from a group consisting of genomic DNA, plasmid DNA, viral DNA, mitochondrial DNA, cDNA and synthetic DNA.

In certain embodiments, the cycling condition of PCR can be modified. Example PCR cycling conditions with modification includes, but not limited to, Touchdown PCR, Stepdown PCR, Slowdown PCR, Nested PCR, Multiple×PCR, Quantitative PCR, Hotstart PCR, Direct PCR and the like.

DNA polymerases commonly used for PCR fall into two families: family A and family B. In exemplary embodiments, both family A (Taq) and family B (KOD) DNA polymerase were tested. In certain embodiments, a variety of thermostable DNA polymerases or their combinations can be used. Useful DNA polymerases include, but are not limited to, KOD DNA polymerase, Taq DNA polymerase, Pfu DNA polymerase, Tli DNA polymerase (also referred as Vent DNA polymerase), Tth DNA polymerase, Tma DNA polymerase, Pfx DNA polymerase, KAPA DNA polymerase, JDF-3 DNA polymerase, Tgo DNA polymerase, GB-D DNA polymerase (also referred to as Deep Vent DNA polymerase). The thermostable DNA polymerases may be mutated or hybridized each other or fused to other amino acid sequences to modify their properties including, but are not limited to, thermostability, polymerase activity, exonuclease activity, proofreading activity, reverse transcription activity, strand displacement activity, substrate binding affinity, fidelity, processivity, elongation rate, sensitivity, specificity, resistance to crude sample, resistance to salt, resistance to chemical compounds, and so on. In certain embodiments, DNA polymerases with one or more of modified properties mentioned above can be used.

In some exemplary embodiments, 2 mM Mg²⁺ in PCR preparation solution for KOD DNA polymerase gave original purplish color and turned to bluish after PCR completion (Example 3, 4, 5, 6). However, 2 mM Mg²⁺ in PCR preparation solution for Taq DNA polymerase, the most popular DNA polymerase for PCR, gave original blue color and did not turned to more bluish after PCR completion. It happened to find that 3.5 mM Mg²⁺ in PCR preparation solution for Taq DNA polymerase overcame the “Taq problem”. In one exemplary embodiment (Example 7), 3.5 mM Mg²⁺ in PCR preparation solution for Taq DNA polymerase was employed. 3.5 mM Mg²⁺ was the minimum concentration required for Taq DNA polymerase to give original purple color before PCR initiation, which then turned to bluish after PCR completion. Therefore, in order to detect PCR amplification by Mg²⁺ indicator, the color of PCR preparation solution needs to be purplish (such as purple or violet) by adjusting the Mg²⁺ concentration. In addition, the Mg²⁺ concentration is preferably at its minimum concentration required to give purplish color before PCR initiation.

In certain embodiments, one or more agents that destabilize nucleic acid interaction are included in a PCR amplification. Examples of agents that destabilize nucleic acid interaction includes, without limitation, Dimethylsulfoxide (DMSO), formamide, betaine, 7-deaza-2′-deoxyguanosine 5′-triphosphate, T4 gene 32 proteins, and the like. Those of ordinary skill in the art may determine the appropriate destabilizing agent and the appropriate concentration for the reaction. Additives such as BSA, non-ionic detergents such as Triton X-100 or Tween 20, NP-40, DTT, and RNase inhibitor may be included for optimization purposes without adversely affecting the amplification reaction. In other embodiment, PCR solutions may be lyophilized to preserve stability for long periods of storage.

This invention provides an easy and reliable way to detect PCR amplification, obviating the need of running gel electrophoresis or sophisticated instrument (such as real time fluorescent PCR machine). The applications of the present invention to detect PCR amplification includes, but not limited to, academic research, diagnostic application, food safety, forensic application, genetic testing. For academic research, the current invention can be used for, but not limited to, colony identification, mouse genetic testing, plant genetic testing, microbe identification for cell culture contamination detection. The robustness of the color change enables efficient detection of target in point-of-care testing or resource-limiting environment. Color change monitored in real time, allows quantification of amount of target nucleic acid where such information is required.

Embodiments of the invention provide a simple means for visual detection of nucleic acid amplification in PCR.

### Example 1

### A Mg²⁺ Indicator in Response to Various Concentration of Mg²⁺ in a PCR Preparation Solution

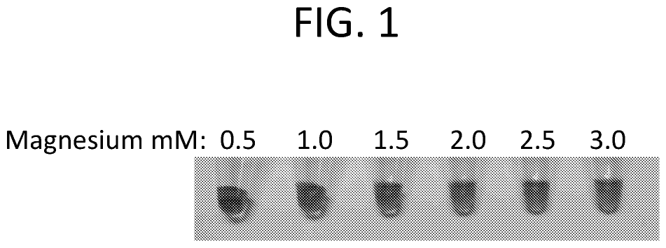

To test whether Mg²⁺ indicator is compatible with PCR preparation solution, EBT was added into PCR preparation solutions containing various concentration of Mg²⁺, and colors were observed by naked eyes without performing PCR reaction (FIG. 1). PCR preparation solutions contained 1×PCR Buffer for KOD Hot Start DNA Polymerase (from EMD Millipore), 0.3 mM dNTPs, 0.12 mM EBT, 0.4 uM forward and reverse primers (SEQ ID, NO: 1 and NO: 2), 0.08 U/ul KOD Hot Start DNA Polymerase (from EMD Millipore) and various concentration of MgSO4 (0.5 mM, 1 mM, 1.5 mM, 2 mM, 2.5 mM and 3 mM). According to this observation, EBT is compatible with PCR preparation solutions and different concentration of Mg²⁺ shows different color. EBT gives rise to different color responsive to Mg²⁺ concentration in a range from 0.5 mM to 2 mM. With the decrease of Mg²⁺ concentration from 2 mM to 0.5 mM, the color of EBT changes from purple to blue. Primer sequences were as follows:

### SEQ NO:1

aacggccaca agttcagcgt gtct

### SEQ NO:2

gcaggaccat gtgatcgcgc ttct

### Example 2

### Detecting Mg²⁺ in a Post-PCR Product by a Mg²⁺ Indicator

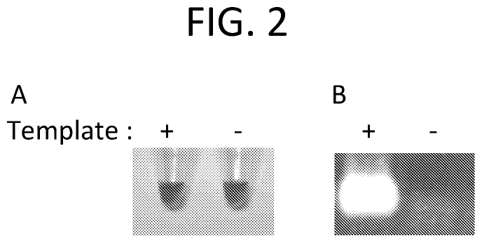

To test whether PCR reaction results in reduction of Mg²⁺ to be detectable by a Mg²⁺ indicator, a PCR reaction was performed, and Mg²⁺ indicator EBT was added into post-PCR product. PCR reactions were performed in 1×PCR Buffer for KOD Hot Start DNA Polymerase (from EMD Millipore), 0.3 mM dNTPs, 2 mM MgSO4, 0.4 uM sense and antisense primers (SEQ ID, NO: 1 and NO: 2), 0.08 U/ul KOD Hot Start DNA Polymerase (from EMD Millipore) in the presence or absence of template DNA (plasmid MigR1, 10⁸ copies). The PCR was performed at 95° C. 2 min, 50 cycles of 95° C. 20 sec and 70° C. 20 sec. After PCR completion, PCR products were supplemented with 0.12 mM EBT to show color (FIG. 2A) and then subjected to gel electrophoresis (FIG. 2B).

The result of FIG. 2 shows that the color of PCR reaction with template was blue and non-template one was purple, which indicates that positive PCR reaction results in reduction of Mg²⁺ concentration. The reduction is significant enough to be detectable by Mg²⁺ indicator EBT. The agarose gel electrophoresis verified that the reaction with blue color had specific amplification, whereas the reaction with purple color did not have any specific amplification. According to the result of Example 1, it is estimated that PCR reaction resulted in Mg²⁺ concentration reduction of 0.5 mM-1 mM. Therefore, Mg²⁺ indicator, such as EBT, successfully detects PCR reaction.

### Example 3

### Sensing PCR by Adding a Mg²⁺ Indicator Prior-to-Reaction

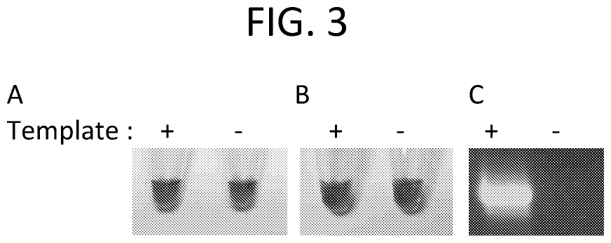

Mg²⁺ indicator EBT was added before starting PCR in this experiment. PCR reaction were performed in 1×PCR Buffer for KOD Hot Start DNA Polymerase (from EMD Millipore), 0.3 mM dNTPs, 2 mM MgSO4, 0.12 mM EBT, 0.4 uM sense and antisense primers (SEQ ID, NO: 1 and NO: 2), 0.08 U/ul KOD Hot Start DNA Polymerase (from EMD Millipore) in the presence or absence of template DNA (plasmid MigR1, 10⁸ copies). As shown in FIG. 3A, the original colors of solutions before PCR initiation were purple. The PCR reactions were performed at 95° C. 2 min, 50 cycles of 95° C. 20 sec and 70° C. 20 sec. After PCR completion, the color of sample containing template DNA (left) turned to blue, whereas the color of non-template control (right) kept its original purple color (FIG. 3B). The agarose gel electrophoresis verified that the reaction with blue color had specific amplification, whereas the reaction kept original purple color did not have any specific amplification (FIG. 3C). The result indicates successful sensing of PCR amplification by a Mg²⁺ indicator EBT added into PCR before initiation of PCR.

### Example 4

### Sensing PCR by a Mg²⁺ Indicator with Different Primers and Different Amount of Template DNA

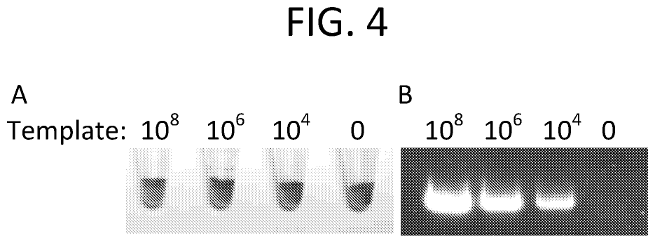

In this experiment, Mg²⁺ indicator EBT was added into PCR preparation solution before PCR initiation, and a pair of primers different from previous examples were employed. PCR reactions were performed in 1×PCR Buffer for KOD Hot Start DNA Polymerase (from EMD Millipore), 0.3 mM dNTPs, 2 mM MgSO4, 0.12 mM EBT, 0.4 uM sense and antisense primers (SEQ ID, NO: 3 and NO: 4), 0.08 U/ul KOD Hot Start DNA Polymerase (from EMD Millipore) in the presence of 10⁸ copies, 10⁶ copies, 10⁴ copies and 0 copy template DNA (MigR1 plasmid) respectively. The PCR was performed at 95° C. 2 min, 60 cycles of 95° C. 20 sec and 70° C. 20 sec. After PCR completion, the photo was taken (FIG. 4A) and all reactions were run agarose gel electrophoresis (FIG. 4B).

As shown, the reaction without template (FIG. 4B, lane 4) had no specific amplification and showed purple color (FIG. 4A, tube 4). In contrast, all reactions with templates had amplifications (FIG. 4B, lane 1,2,3) and showed blue color (FIG. 4A, tube 1,2,3). Therefore, positive amplifications gave blue color, whereas negative amplification gave purple color. The result re-confirmed the successful detection of PCR amplification by a Mg²⁺ indicator. Primer sequences were as follows:

### SEQ NO:3

tgagcaaggg cgaggagctg tt

### SEQ NO:4

gcgaacagaa gcgagaagcg aactgatt

### Example 5

### Real-Time Sensing of PCR by Mg²⁺ Indicator

In this experiment, the colors of Mg²⁺ indicator were monitored during PCR in a real-time manner. The same PCR reactions as Example 4 were performed. After 25 cycles, the colors of samples were visually observed continuously for each cycle. To minimize the influence of temperature disturbance resulting from repeated lid opening of PCR machine, the colors of reactions were observed at end of denature step (95° C. 20 sec) of each cycle. For a given reaction, its threshold number of cycles was thus recorded. The threshold number of cycles here is defined as the number of cycles at which a visible color difference is observed between template-containing samples and non-template control. As shown in Table 1, the threshold number of cycles reversely correlated to template amount. More cycles are needed for reactions with less template DNA to change color. The non-template control kept its purple color during the whole PCR without obvious color change. The threshold number of cycles therefore provide a way to quantify the template.

TABLE 1
The threshold number of cycles of color change in response to
different template amount

| | Threshold number of cycles for |
|---|---|
| Template (copies) | EBT color changing |
| 10⁸ | 34 |
| 10⁶ | 48 |
| 10⁴ | 59 |
| 0 | No color change |
| | detected |

### Example 6

### Sensing PCR by a Different Mg²⁺ Indicator

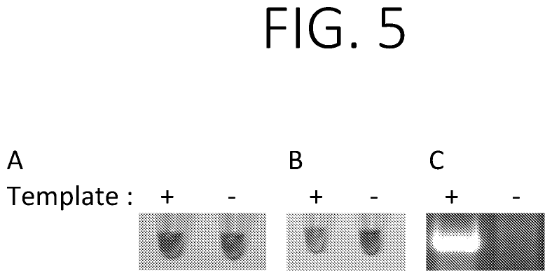

Another magnesium-sensitive dye, HNB, was tested here for its ability to sense PCR. PCR reactions was performed in 1×PCR Buffer for KOD Hot Start DNA Polymerase (from EMD Millipore), 0.3 mM dNTPs, 2 mM MgSO4, 0.12 mM HNB, 0.4 uM sense and antisense primers (SEQ ID, NO: 3 and NO: 4), 1 mM Sodium Citrate, 0.08 U/ul KOD Hot Start DNA Polymerase (from EMD Millipore) in the presence and the absence of 10⁸ copies template DNA (MigR1 plasmid) respectively. The PCR was performed at 95° C. 2 min, 50 cycles of 95° C. 20 sec and 70° C. 20 sec. Before initiation of PCR, all solutions are violet color (FIG. 5A). After PCR completion, the template-containing reaction (left) turned to sky-blue color and the non-template reaction (right) kept its violet color (FIG. 5B). All samples were run agarose gel electrophoresis (FIG. 5C). As shown, the reaction containing template DNA changed its original violet to sky-blue, and its amplification was verified by agarose gel electrophoresis. The result indicated successful detection of PCR amplification by Mg²⁺ indicator HNB.

### Example 7

### Sensing PCR by a Mg²⁺ Indicator with Taq DNA Polymerase

Taq DNA polymerase was tested here in this experiment. EBT was added before the start of PCR. PCR reaction was performed in 1× Thermopol Buffer (from NEB, containing 2 mM Mg²⁺), 0.3 mM dNTPs, additional 1.5 mM MgSO4, 0.12 mM EBT, 0.4 uM sense and antisense primers (SEQ ID, NO: 3 and NO: 4), 0.2 U/ul Taq (from New England Biolabs) in the presence or absence of 10⁸ copies of template DNA (MigR1 plasmid) respectively. The PCR was performed at 95° C. 2 min, 60 cycles of 95° C. 20 sec and 70° C. 70 sec. Hotstart procedure was manually employed. Taq DNA polymerase was added during the initial denature step (95° C. 2 min) of PCR.

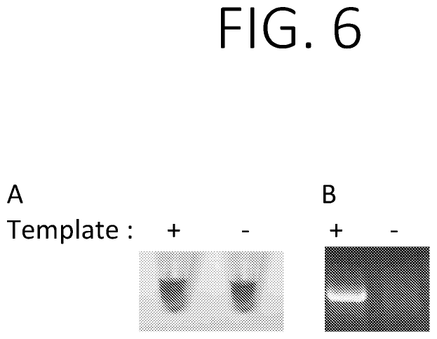

After PCR completion, the photo of the reactions was taken (FIG. 6A). As shown, positive reaction with template (left) changed to bluish, which indicates the reduction of Mg²⁺, whereas no-template reaction (right) kept its original purple color. The agarose gel electrophoresis of the two reactions confirmed that the positive reaction had amplification (FIG. 6B). The result indicated that PCR sensing by Mg²⁺ indicator works for PCR with Taq DNA polymerase.

1. An aqueous preparation for PCR reaction comprising: a magnesium-sensitive colorimetric dye, a buffer, magnesium ion, primers, a DNA polymerase and dNTPs.

2. A preparation according to claim 1, wherein the PCR reaction further comprises template DNA.

3. A preparation according to claim 1, wherein the dye is a visually detectable color dye.

4. A preparation according to claim 3, wherein the dye is selected from Eriochrome Black T, Hydroxynaphthol Blue.

5. A post-PCR reaction comprising a magnesium-sensitive colorimetric dye, wherein the dye is added before initiation of PCR or after PCR completion.

6. A post-PCR reaction according to claim 5, wherein the dye is a visually detectable color dye.

7. A post-PCR reaction according to claim 6, wherein the dye is selected from Eriochrome Black T, Hydroxynaphthol Blue.

8. A method for detecting PCR amplification; comprising:
providing a PCR amplification reaction mixture comprising a magnesium-sensitive colorimetric dye, a buffer, magnesium ion, a DNA polymerase, dNTPs, primers and template DNA;
and
detecting a color change of the dye resulting from amplification of the target DNA.

9. A method according to claim 8, wherein the dye is added before initiation of PCR.

10. A method according to claim 8, wherein the dye is added after PCR completion.

11. A method according to claim 8, wherein the dye is a visually detectable color dye.

12. A method according to claim 11, wherein the dye is selected from Eriochrome Black T, Hydroxynaphthol Blue.

13. A method according to claim 9, wherein the color of the dye is monitored during PCR reaction.

14. A method according to claim 13, further comprising quantification of template DNA by threshold number of cycles.

15. A method according to claim 9, further comprising comparing a color change of the magnesium-sensitive dye from before to after the PCR reaction.

16. A method according to claim 9, further comprising comparing the colors of PCR reactions with template to that of PCR reactions without template.

17. A method according to claim 10, further comprising comparing the colors of PCR reactions with template to that of PCR reactions without template.


SEQUENCE LISTING (4 sequences)

SEQ ID NO: 1 - DNA, 24 bp, jellyfish Aequorea Victoria
aacggccacaagttcagcgtgtct

SEQ ID NO: 2 - DNA, 24 bp, jellyfish Aequorea Victoria
gcaggaccatgtgatcgcgcttct

SEQ ID NO: 3 - DNA, 22 bp, jellyfish Aequorea Victoria
tgagcaagggcgaggagctgtt

SEQ ID NO: 4 - DNA, 28 bp, jellyfish Aequorea Victoria
gcgaacagaagcgagaagcgaactgatt